(19) United States
(12) Patent Application Publication
(10) Pub. No.: US 2024/0029875 A1
(43) Pub. Date: Jan. 25, 2024

(54) SYSTEM AND METHOD TO RECOMMEND SERVICE ACTION FOR PREDICTIVE MAINTENANCE

(71) Applicant: KONINKLIJKE PHILIPS N.V., EINDHOVEN (NL)

(72) Inventors: Ravindra PATIL, BANGALORE (IN); Michael Leonardus Helena BOUMANS, NEDERWEERT-EIND (NL)

(21) Appl. No.: 18/265,996

(22) PCT Filed: Dec. 9, 2021
(86) PCT No.: PCT/EP2021/084876

§ 371 Date: Jun. 8, 2023

Related U.S. Application Data
(60) Provisional application No. 63/125,541, filed on Dec. 15, 2020.

(51) Int. Cl. G16H 40/40; G16H 50/70; G06N 5/022

(52) U.S. Cl. CPC G16H 40/40; G16H 50/70; G06N 5/022

(57) ABSTRACT

A non-transitory computer readable medium (107, 127) stores: a predictive model (130) configured to generate an alert (132) predicting a failure of a component of a medical imaging device (120) by applying patterns to values of a set of features; a table (136) having records corresponding to the patterns of the predictive model; and instructions readable and executable by at least one electronic processor (101, 113) to (i) train: a sequence model (134) to receive values of the set of features for a current case and to output a most probable root cause and at least one service action for the current case, and (ii) determine a root cause and at least one recommended service action for the alert generated by the predictive model by applying the trained sequence model to the values of the set of features for the medical imaging device.

### FIELD

The following relates generally to the medical device maintenance arts, predictive maintenance arts, service action recommendation arts, and related arts.

### BACKGROUND

Medical devices undergo numerous maintenance activities during their life span, such as calibrating parts, lubrication of parts, minor repairs, etc. Failure of a particular part or component of a medical imaging device can result in downtime of the imaging device which costs the hospital (or other medical facility) financially as the imaging device is not generating revenue, and is also costly in terms of patient dissatisfaction if, for example, a patient's imaging examination must be rescheduled. Much of these costs are due to the failure being unanticipated. Downtime due to a failing part or component can be reduced or eliminated if the impending failure is proactively identified so that the repair can be done as scheduled maintenance, enabling the repair to be done during hours when the medical imaging device is not in use, or at least enabling the hospital to adjust its schedule to accommodate the maintenance. To this end, it is known to provide predictive failure models that predict when components are likely to fail. However, these predictive models typically do not provide information as to the type of service action that is needed to remediate the predicted failure.

In some service activities, a service engineer (SE, which can be a field service engineer (FSE) or a remote service engineer (RSE)) replaces particular part or set of parts that are deemed not fit for further usage. However, the predicted failure of a component might correlate to different causes of the failures, requiring different service action for the same particular component failure to fix the issue. For example, a “touch screen module” failure in an image-guided therapy (iGT) system may be due to the connected cables malfunction, or may be due to the failure of the touch screen buttons. The former requires a low-cost cable replacement; whereas the latter may require a more costly replacement of the entire touch screen module. However, the predictive failure model typically provides only the “touch screen module” failure prediction.

Hence, it would be advantageous to automatically detect what service action most likely needs to be performed, and to provide an automatic recommendation of that service action, when the cause of failures might be multiple for the same component failure. Such a service action recommender could provide numerous benefits, such as optimized warehousing, facilitating first time successful repair and consequent high customer satisfaction, and reducing the time spent by an FSE to diagnose and fix the issue.

The following discloses certain improvements to overcome these problems and others.

### SUMMARY

In one aspect, a non-transitory computer readable medium stores a predictive model configured to generate an alert predicting a failure of a component of a medical imaging device by applying patterns to values of a set of features for the medical imaging device obtained from a log automatically generated by the medical imaging device. A table has records corresponding to the patterns of the predictive model and fields for each record including (i) at least one field storing the features of the set of features that are used in the pattern, (ii) a field storing a root cause associated with the pattern, and (iii) a field storing at least one recommended service action associated with the pattern. Instructions are readable and executable by at least one electronic processor to train a sequence model to receive values of the set of features for a current case and to output a most probable root cause and at least one service action for the current case, the training being on data for historical cases in which the data for each historical case includes values for the fields of the table. Instructions are readable and executable by the at least one electronic processor to determine a root cause and at least one recommended service action for the alert generated by the predictive model by applying the trained sequence model to the values of the set of features for the medical imaging device.

In another aspect, a non-transitory computer readable medium stores a predictive model configured to generate an alert predicting a failure of a component of a medical imaging device by applying patterns to values of a set of features for the medical imaging device obtained from a log automatically generated by the medical imaging device. A table has records corresponding to the patterns of the predictive model and fields for each record including (i) at least one field storing the features of the set of features that are used in the pattern, (ii) a field storing a root cause associated with the pattern, and (iii) a field storing at least one recommended service action associated with the pattern. Instructions are readable and executable by at least one electronic processor to train a sequence model comprising a Hidden Markov Model (HMM) to receive values of the set of features for a current case and to output a most probable root cause and at least one service action for the current case, the training being on data for historical cases in which the data for each historical case includes values for the fields of the table. Instructions readable and executable by the at least one electronic processor to determine a root cause and at least one recommended service action for the alert generated by the predictive model by applying the trained sequence model to the values of the set of features for the medical imaging device.

In another aspect, a service device includes a display device; at least one user input device; at least one electronic processor, and a non-transitory storage medium storing instructions readable and executable by the at least one electronic processor to determine a root cause and at least one recommended service action for an alert predicting a failure of a component of a medical imaging device. The alert is generated by a predictive model by applying a trained sequence model to values of a set of features for the medical imaging device.

One advantage resides in augmenting a failure prediction model with service action recommendation for automatic identification of a service action needed to be performed for a failing component of a medical device.

Another advantage resides in augmenting a failure prediction model with a modeling system for identifying a most likely root cause of failure for a predicted failure of a component of a medical device, and a corresponding best service action to be performed.

Another advantage resides in providing a modeling system for predicting a service action to be performed by a FSE on a medical device, thereby reducing the amount of time spent by the FSE during a service call.

Another advantage resides in providing an automatic service action recommendation without manual intervention for service action to be performed by a FSE on a medical device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for determining a root cause and at least one recommended service action for servicing a medical device in accordance with the present disclosure.

FIG. 2 presents a portion of a table having records corresponding to the patterns of the predictive models, used as an illustrative example herein.

FIG. 3 shows exemplary flow chart operations of the system of FIG. 1.

FIGS. 4 and 5 show illustrative outputs generated by the system of FIG. 1.

### DETAILED DESCRIPTION

Predictive failure models are run on imaging device machine log data to provide predictions of when components are likely to fail. These models are typically machine learning (ML) models that are trained on historical data to proactively predict how likely a component is to fail, and/or may be constructed manually or semi-manually by domain experts based on historical data and/or a priori knowledge. Typically, the predictive model operates by applying patterns to values of a set of features for the medical imaging device obtained from a log that is automatically generated by the medical imaging device. For example, a predictive model for predicting an X-ray tube failure may apply: (1) a first pattern that detects the current time is more than a predefined time interval since of an X-ray tube was installed; and (2) a second pattern that detects the X-ray tube current has been increasing over time. Both patterns may again trigger an alert that an X-ray tube failure may occur soon—but the root causes and service actions may be quite different. The first pattern detects the X-ray tube is nearing its end-of-useful-life so that the root cause is likely to be tube failure and the appropriate service action is tube replacement. By contrast, the second pattern detects may have a root cause of degraded electrical contacts and the appropriate service action may be to clean the contacts of the X-ray tube socket and, if this does not solve the problem, to replace the X-ray tube socket. However, the predictive models are typically only trained to predict the failure, and do not generally provide identification of a likely root cause or service action guidance.

To provide this additional information (i.e., likely root cause and/or service action recommendation), the following discloses constructing a table with a row (more generally, a record) for each pattern of each predictive model that can produce a component failure alert. Each row includes the following columns (more generally, fields): Model, required features to produce the alert, other features (optional), root cause, and service action. The table may be manually constructed, or automatically constructed by mining the relevant information from fault finding trees or other diagnostic flowcharts provided in service manuals.

Next, a sequence model is trained on historical cases that are annotated with the root cause and service action determined in the resolution of the respective cases. For each row of the table, the weights of the features are extracted from the predictive model. These weights may be the actual weights applied to the features in the model, or these weights may be obtained by feature importance analysis in which the sensitivity of the model to the various features is determined by iteratively running the predictive model with feature values varied. For each row and for each historical case, an input sequence is constructed which includes the model type, the features used, and the weights of the features; and a corresponding output dataset includes the root cause and service action(s). The historical cases form a “sequence of sequences” which serve as input training data for training a sequence model. A Hidden Markov Model (HMM) is used as the sequence model in the illustrative examples presented herein, but other sequence models can be used such as a Gaussian Mixture Model (GMM) or a Long-Term Short Memory (LSTM) model. The sequence model is trained until its parameters reach a steady state.

Thereafter, the trained sequence model can be used as follows. When a predictive model issues an alert for a particular imaging device predicting a component failure, the input sequence of the trained sequence model is constructed for the particular imaging device using values drawn from the machine log for the particular imaging device, and this input sequence is fed into the trained sequence model which outputs the most likely root cause and service action(s).

In use, the disclosed system can be integrated into the proactive alerting system as follows. When an alert is issued by a predictive model, the system is automatically invoked to identify the most likely root cause and service action(s). The alert is then presented to a Remote Service Engineer (RSE) or a field service engineer (FSE) (or, more generally, a service engineer, i.e., “SE”) along with the most likely root cause and the recommended service actions.

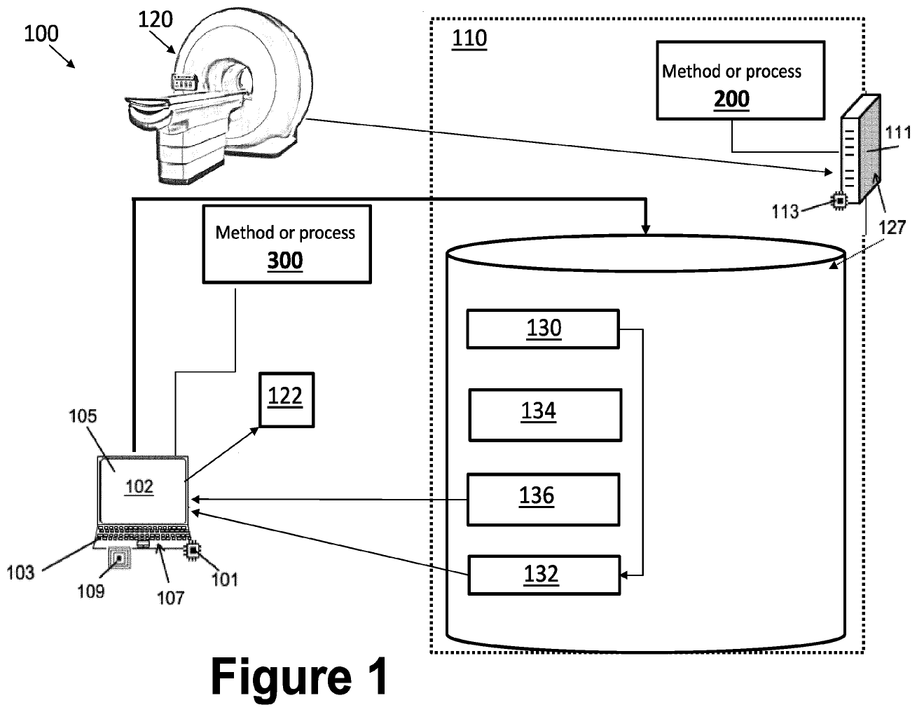

With reference to FIG. 1, an illustrative servicing support system 100 for supporting a service engineer in servicing a device 120 (e.g., a medical imaging device—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device that automatically generates log data that are analyzed by predictive models to predict component failures, e.g., the approach could be applied to a commercial airliner, radiation therapy device, or so forth). As shown in FIG. 1, the servicing support system 100 includes, or is accessible by, a service device 102 that may for example be a workstation used by a RSE. In another example, the service device 102 may be a portable device such as a notebook computer that is carried or accessed by an FSE. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by a FSE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer.

The service device 102 includes a display 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is a RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or other servicing contractor, or by the medical facility that owns or leases the imaging device 120). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling and (as disclosed herein) root cause and service recommendation analyses is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for each such medical imaging device.

With continuing reference to FIG. 1, the non-transitory computer readable medium 127 stores one or more predictive models 130. The predictive models 130 are configured to generate an alert 132 configured to predicting a failure of a component of the medical imaging device by applying patterns to values of a set of features for the medical imaging device obtained from a log (not shown) automatically generated by the medical imaging device 120. The log is transferred from the medical imaging device 120 to the backend server 111.

The non-transitory computer readable medium 127 also stores one or more sequence models 134 configured to output a most probable root cause and at least one service action for a current service case for the medical imaging device 120 by an FSE. The sequence models 134 can be, for example, a Hidden Markov Model (HMM), a Gaussian Mixture Model (GMM), a Long Short-Term Memory (LSTM) model, or any other suitable sequence model.

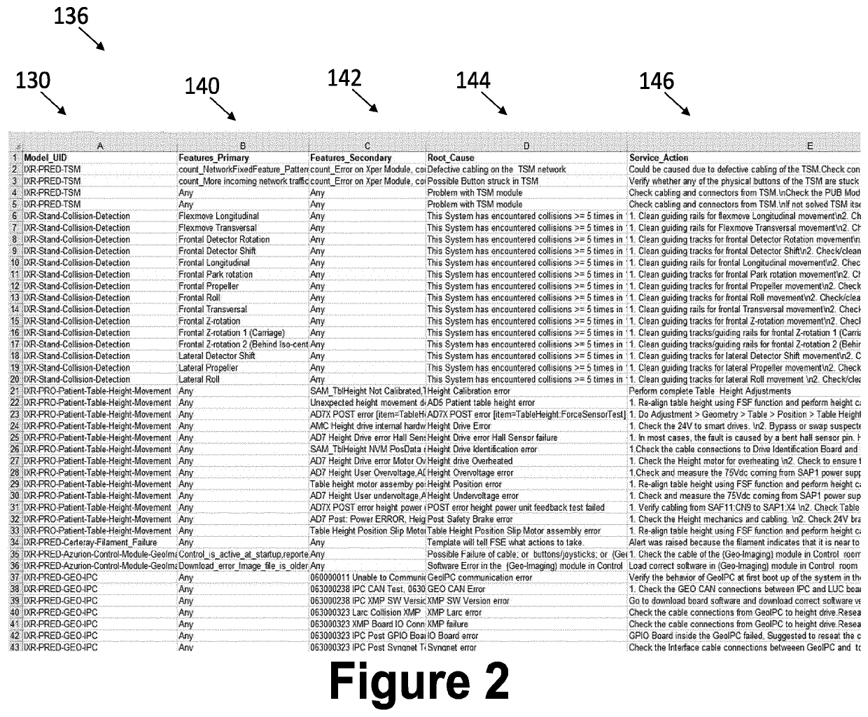

The non-transitory computer readable medium 127 further stores a table 136 having records corresponding to the patterns of the predictive models 130. As used herein, the term “record” (and variants thereof) refers to a “row” of the table 136. The table 136 also includes one or more fields for each record. As used herein, the term “field” (and variants thereof) refers to a “column” of the table 136. An example of the table 136 is depicted in FIG. 2.

FIG. 2 shows a portion of an exemplary table 136. The table of FIG. 2 is constructed in the Microsoft Excel spreadsheet program (available from Microsoft Corporation, Redmond, WA, USA); however, another spreadsheet program such as that provided by the LibreOffice office suite could be used to construct the table 136. Furthermore, the skilled artisan will recognize that the Excel spreadsheet table 136 shown in FIG. 2 has manually adjustable column width display, and that text of some of the fields is truncated by the chosen column widths of the various columns. As shown in FIG. 2, the fields (i.e., columns) of the table 136 can include, for example, includes (from left to right) a field storing an identification of the predictive model 130, a field 140 storing primary features of the set of features that are used in the pattern to generate the alert 132, a field 142 storing secondary features of the set of features that are used in the pattern to generate the alert 132, a field 144 storing a root cause associated with the pattern, and a field 146 storing at least one recommended service action associated with the pattern. The table 136 may not be limited to these fields, and can include additional or fewer fields.

The non-transitory storage medium 127 stores instructions executable by the electronic processor 113 of the backend server 111 to perform a training method 200 of training the sequence models 134 to receive values of the set of features for a current case (i.e., a maintenance case on the medical imaging device 120 performed by the FSE) and to output a most probable root cause and at least one service action for the current case. The training method 200 can be performed with on data for historical cases in which the data for each historical case includes values for the fields of the table 136. The table 136 is used during the training of the sequence model 134, such as for when the predictive model 130 might have been triggered due to multiple features combined. The sequence model 134 is built to identify different relationships among these features and arrives at which are the primary and secondary features that were responsible for the trigger of the predictive model 130. Based on the combination of different features, the sequence model 134 is trained to provide an appropriate root cause and solution.

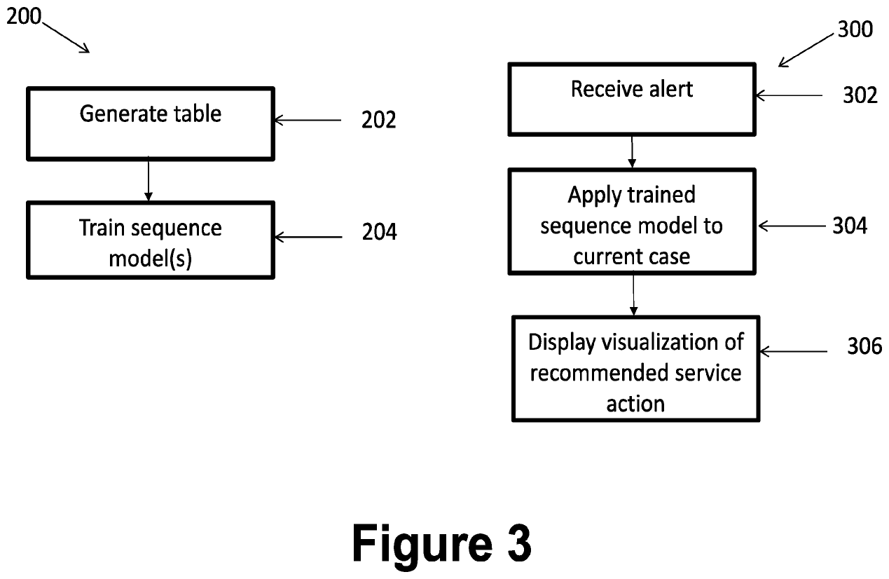

With continuing reference to FIG. 1 and further reference to FIG. 3, an illustrative embodiment of the training method 100 executable by the electronic processor 113 is diagrammatically shown as a flowchart in the left-hand side of FIG. 3. In some examples, the method 200 may be performed at least in part by cloud processing.

To begin the training method 200, at an operation 202, the table 136 is generated. In one embodiment, the table is generated by the backend server 111 by mining data from service manuals of the medical imaging device 120 and/or from one or more databases (e.g., the non-transitory storage medium 127). In another embodiment, a graphical user interface (GUI) 122 can be provided on the display device 105 of the service device 102, and the table 134 can be input by the FSE via the GUI and stored in the backend server 111.

At an operation 204, a training operation 204 is performed to train the sequence model 134. In some embodiments, the training operation 204 includes extracting weights for the features in the sequence model 134 based on the at least one field storing the features of the set of features that are used in the pattern applied to the predictive model 130. For example, the weights can be extracted for the features in the sequence model 134 based on weights of the features in the predictive model 130. In another example, the weights for the features in the sequence model 134 can be obtained a feature importance analysis, which can include determining a sensitivity of the predictive model 130 to the features by running the predictive models with varied feature values.

Although described in terms of a single predictive model 130 and a single sequence model 134, the training method 200 can be applied for multiple predictive models (for example, Table 1 shows multiple predictive models) and multiple sequence models. For example, the various components of the medical imaging device 120 suitably have corresponding respective predictive failure models 130.

With continuing reference to FIGS. 1-3, an illustrative embodiment of an instance of service action recommendation method 300 executable by the electronic processor 101 is diagrammatically shown as a flowchart in FIG. 3 (e.g., on the right-hand side of FIG. 3). The service action recommendation method 300 includes determining a root cause of the failure that is the subject of the current maintenance case of the medical imaging device 120 serviced by the FSE, and at least one recommended service action for the alert 132 generated by the predictive model 130 by applying the trained sequence model to the values of the set of features for the medical imaging device.

To begin the method 300, at an operation 302, the service device 102 receives the alert 132 generated from the predictive model 130 from the backend server 111 of the failure that is the subject of the current maintenance case of the medical imaging device 120 serviced by the FSE. The alert 132 can be visually displayed on the GUI 122, audibly output by a speaker (not shown) of the service device 102, and so forth.

At an operation 304, the backend server 111 applies the trained sequence model 134 to the features of the current case to generate the likely root cause and recommended service action(s). At an operation 306, a visualization of the table 136 can be displayed on the display device 105 of the service device based on the correlation between the trained sequence model 134 and the features of the current case. The visualization of the table 136 can show for example, the root cause field and the recommended action field. The FSE can then address the alert 132 by performing the recommended service action shown on the display device 105.

### Examples

The following describes examples of the training of the sequence model 134. To train the sequence model 134, an interaction of different features and the weight derived from each feature to arrive at the best possible root cause (from the table 136). All the features that are responsible for triggering the alert 132 are considered and are weighted to find the contribution of each of the features. Each of the feature weights are appropriately adapted to predict the most appropriate service action.

The features can include, for example, model type, different features being used, contribution of each features, the recommendation is identified using the sequence model 134 as an HMM. The objective of the HMM is given the sequence of parameters it is trained to find the best root cause and service action(s). A second order Hidden Markov Model (HMM) is trained on data for historical cases, which includes values for a set of features that includes the features required by the predictive failure model (corresponding to the “Features_Primary” column 140 of the table 136 of FIG. 2 for all patterns of the model) and optionally also additional features (e.g., the features listed in the “Features_Secondary column 142 of the table 136). These values are suitably obtained from a log for the medical imaging device that is automatically generated by the medical imaging device. Denoting the set of values for the historical case indexed by i as xᵢ, if there are n historical cases then this provides a sequence X=x₁, x₂, . . . , xₙ. Each historical case i is also annotated with the root cause and service action(s) determined for that case—this information is denoted as yᵢ for the case i, so that the sequence X has a corresponding output sequence Y=y₁, y₂, . . . , yₙ. It is then desired to maximize the probability:

p(x₁,x₂, . . . ,xₙ,y₁,y₂, . . . ,yₙ) (a)

Using the HMM formulation, the most likely service action and efficiency for X is:

$${argmax}_{{y}_{1} , {y}_{2} , … , {y}_{n}} p ( {x}_{1} , {x}_{2} , … , {x}_{n} , {y}_{1} , {y}_{2} , … , {y}_{n} ) \qquad ( b )$$

where:

$$p ( {x}_{1} , {x}_{2} , … , {x}_{n} , {y}_{1} , {y}_{2} , … , {y}_{n} ) = {\prod }_{i = 1}^{n + 1} q ( {y}_{i} \vert {y}_{i - 2} , {y}_{i - 1} ) {\prod }_{i = 1}^{n} e ( {x}_{i} \vert {y}_{i} ) \qquad ( c )$$

Here q(x|y) are the transition probabilities between states, and e (x|y) are the emission probabilities. The probabilities q(x|y) and e(x|y) are suitably estimated using standard maximum likelihood estimation techniques. Finally, to perform the operation 304 of FIG. 3, given a set of values xᵢₙₚᵤₜ for a medical imaging device 120 that has received an alert 132 generated by the predictive model 130 (also referred to herein as a “current service case”), the likelihood of a possible output sequence yₒᵤₜₚᵤₜ denoting a possible combination of root cause and service action(s) is given by:

p(xᵢₙₚᵤₜ,yₒᵤₜₚᵤₜ)=e(xᵢₙₚᵤₜ|yₒᵤₜₚᵤₜ) (d)

Equation (d) uses the values of the probabilities q(x|y) and e (x|y) as optimized for the historical cases using Equations (b) and (c). The output sequence yₒᵤₜₚᵤₜ for which probability p(xᵢₙₚᵤₜ, yₒᵤₜₚᵤₜ) is highest can be chosen as the root cause and recommended service action(s), or alternatively the possible output sequences can be ranked by their respective probabilities p(xᵢₙₚᵤₜ, yₒᵤₜₚᵤₜ) and, for example, the top two or top three root causes and recommended service action(s) may be reported.

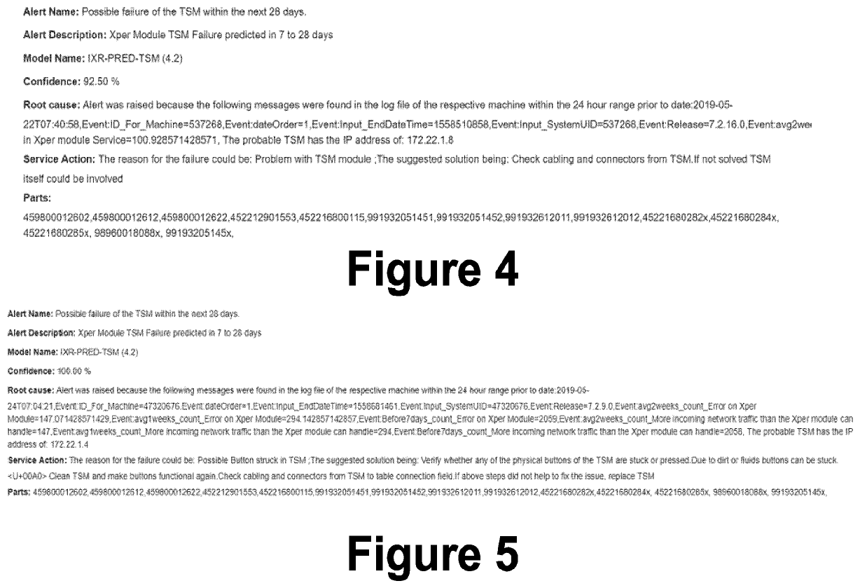

With reference to FIGS. 4 and 5, two illustrative examples of output suitably generated by the servicing support system 100 and displayed on the display 105 of the service device 102 are shown. In both cases, the alert is “Possible failure of the TSM within the next 28 days”. (The acronym “TSM” stands for a table-side module of the medical imaging device 120). However, in the example of FIG. 4, the likely root cause (i.e., “reason for failure”) was determined by the method 300 to be a problem with the TSM module, and the corresponding service action recommendation was “Check cabling and connectors from TSM. If not solved TSM itself could be involved.” By contrast, in the example of FIG. 5, the likely root cause was determined by the method 300 to be a possible button stuck in TSM, and the corresponding service action recommendation was “Verify whether any of the physical buttons of the TSM are stuck or pressed.” With the provided likely root cause and service action recommendation, the remote service engineer is better prepared to address the problem, either by instructing hospital personnel to perform the recommended service action or sending out an FSE with the appropriate part(s) for implementing the service action recommendation.

As used herein, the term “service action” can refer to performing some test, calibrating a subsystem, lubricating, or cleaning some part(s), et cetera. It may also involve the replacement of a part and subsequent test whether or not this solved the issue. Note that the duration of replacing a part will greatly depend on whether or not the FSE currently has a spare example of this part. If not, then replacing might cost one or more days to order and deliver the spare part. Advantageously, the service action recommendation provides information by which the FSE can bring the likely spare part, thereby avoiding this potential delay.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a predictive model configured to generate an alert predicting a failure of a component of a medical imaging device by applying patterns to values of a set of features for the medical imaging device obtained from a log automatically generated by the medical imaging device;
a table having records corresponding to the patterns of the predictive model and fields for each record including (i) at least one field storing the features of the set of features that are used in the pattern, (ii) a field storing a root cause associated with the pattern, and (iii) a field storing at least one recommended service action associated with the pattern; and
instructions readable and executable by at least one electronic processor to train a sequence model to receive values of the set of features for a current case and to output a most probable root cause and at least one service action for the current case, the training being on data for historical cases in which the data for each historical case includes values for the fields of the table; and
instructions readable and executable by the at least one electronic processor to determine a root cause and at least one recommended service action for the alert generated by the predictive model by applying the trained sequence model to the values of the set of features for the medical imaging device.

2. The non-transitory computer readable medium of claim 1, wherein the fields for each record further include a field storing an identification of the predictive model.

3. The non-transitory computer readable medium of claim 1, wherein the training comprises:
extracting weights for the features in the sequence model based on the at least one field storing the features of the set of features that are used in the pattern.

4. The non-transitory computer readable medium of claim 1, wherein the training includes:
extracting weights for the features in the sequence model based on weights of the features in the predictive model.

5. The non-transitory computer readable medium of claim 1, wherein the training includes:
obtaining weights for the features in the sequence model from a feature importance analysis.

6. The non-transitory computer readable medium of claim 1, wherein the sequence model comprises a Hidden Markov Model (HMM).

7. The non-transitory computer readable medium claim 1, wherein the sequence model comprises a Gaussian Mixture Model (GMM).

8. The non-transitory computer readable medium of claim 1, wherein the sequence model comprises a Long Short-Term Memory (LSTM) model.

9. The non-transitory computer readable medium of claim 1, further storing instructions readable and executable by at least one electronic processor to:
providing a user interface via which the table is input via at least one user input device.

10. The non-transitory computer readable medium of claim 1, further storing instructions readable and executable by at least one electronic processor to:
generate the table by mining data from service manuals of the medical imaging device and/or from one or more databases.

11. A non-transitory computer readable medium storing:
a predictive model configured to generate an alert predicting a failure of a component of a medical imaging device by applying patterns to values of a set of features for the medical imaging device obtained from a log automatically generated by the medical imaging device;
a table having records corresponding to the patterns of the predictive model and fields for each record including (i) at least one field storing the features of the set of features that are used in the pattern, (ii) a field storing a root cause associated with the pattern, and (iii) a field storing at least one recommended service action associated with the pattern; and
instructions readable and executable by at least one electronic processor to train a sequence model comprising a Hidden Markov Model (HMM) to receive values of the set of features for a current case and to output a most probable root cause and at least one service action for the current case, the training being on data for historical cases in which the data for each historical case includes values for the fields of the table; and
instructions readable and executable by the at least one electronic processor to determine a root cause and at least one recommended service action for the alert generated by the predictive model by applying the trained sequence model to the values of the set of features for the medical imaging device.

12. The non-transitory computer readable medium of claim 11, wherein the fields for each record further include a field storing an identification of the predictive model.

13. The non-transitory computer readable medium of claim 11, wherein the training comprises:
extracting weights for the features in the sequence model based on the at least one field storing the features of the set of features that are used in the pattern.

14. The non-transitory computer readable medium of claim 11, wherein the training includes:
extracting weights for the features in the sequence model based on weights of the features in the predictive model.

15. The non-transitory computer readable medium of claim 11, wherein the training includes:
obtaining weights for the features in the sequence model from a feature importance analysis.

16. The non-transitory computer readable medium of claim 11, further storing instructions readable and executable by at least one electronic processor to:
providing a user interface via which the table is input via at least one user input device.

17. The non-transitory computer readable medium of claim 11, further storing instructions readable and executable by at least one electronic processor to:
generate the table by mining data from service manuals of the medical imaging device and/or from one or more databases.

18. A service device, comprising:
a display device;
at least one user input device; and
at least one electronic processor; and
a non-transitory storage medium storing instructions readable and executable by the at least one electronic processor to determine a root cause and at least one recommended service action for an alert predicting a failure of a component of a medical imaging device, the alert being generated by a predictive model by applying a trained sequence model to values of a set of features for the medical imaging device.

19. The service device of claim 18, wherein the trained sequence model is trained to receive values of the set of features for a current case and to output a most probable root cause and at least one service action for the current case.

20. The service device of claim 18, wherein the sequence model comprises a Hidden Markov Model (HMM).
